(19) United States
(12) Patent Application Publication
(10) Pub. No.: US 2014/0005948 A1
(43) Pub. Date: Jan. 2, 2014

(54) METHOD AND APPARATUS FOR PERFORMING QUANTITATIVE ANALYSIS OF NUCLEIC ACID USING REAL-TIME PCR

(71) Applicant: SAMSUNG ELECTRONICS CO., LTD., Suwon-si (KR)

(72) Inventors: Soo-kwan LEE, Seoul (KR); Kyung-ho KIM, Seoul (KR); Joon-ho KIM, Seongnam-si (KR); Sun-ok JUNG, Seongnam-si (KR); Kak NAMKOONG, Seoul (KR)

(73) Assignee: SAMSUNG ELECTRONICS CO., LTD., Suwon-si (KR)

(21) Appl. No.: 13/846,456

(22) Filed: Mar. 18, 2013

(30) Foreign Application Priority Data
Jun. 28, 2012 (KR) 10-2012-0070235

(51) Int. Cl. G01N 33/48

(52) U.S. Cl. CPC G01N 33/48
USPC 702/19

(57) ABSTRACT

A method and apparatus for performing quantitative analysis of a nucleic acid by determining a curve-fitting area based on fluorescence intensity data obtained by performing PCR on a target nucleic acid; analyzing parameters for amplification efficiency and nucleic acid concentration by curve-fitting a result of performing PCR on a reference nucleic acid with a known initial nucleic acid concentration; and estimating the initial nucleic acid concentration of the target nucleic acid by performing curve-fitting on the determined curve-fitting area using the analyzed parameters.

### CROSS-REFERENCE TO RELATED APPLICATIONS

This application claims the benefit of Korean Patent Application No. 10-2012-0070235, filed on Jun. 28, 2012, in the Korean Intellectual Property Office, the entire disclosure of which is hereby incorporated by reference.

### BACKGROUND

1. Field

The present disclosure relates to methods and apparatuses for performing quantitative analysis of nucleic acid using real-time polymerase chain reaction (PCR).

2. Description of the Related Art

Polymerase chain reaction (PCR) is a technology used to amplify a small sample of nucleic acid by several orders of magnitude, generating many copies of a particular nucleic acid sequence. PCR may be used to amplify nucleic acids, such as human DNA, to diagnose various genetic disorders. Furthermore, PCR may be used to amplify nucleic acids of bacteria, viruses, and fungi to diagnose infectious diseases.

Generally, PCR consists of three stages including (1) a denaturation stage for separating nucleic acid strands from each other using heat, (2) an annealing stage in which the temperature is lowered and primers bind to an end of a nucleic acid sequence to be amplified, and (3) a polymerization (or extension) stage in which a polymerization reaction is induced to synthesize nucleic acid. When one cycle of PCR is performed, the amount of nucleic acid in the sample is doubled. Therefore, by performing repeated real-time PCR cycles, the amount of nucleic acid in the sample is amplified by geometric progression.

Recently, real-time PCR has been used for quantitative analysis of nucleic acids. Real-time PCR data are typically produced as a sigmoidal-shaped amplification plot in which fluorescence is plotted against the number of cycles. A cycle threshold (C_{T}) value serves as a tool for calculation of the starting template amount in a sample (i.e., quantification of the initial concentration of nucleic acid in a sample). A C_{T} may be obtained by using a threshold value method of determining an x-axis value crossing the sigmoid curve representing fluorescence intensity by setting an arbitrary line parallel to the x-axis. Alternatively, a C_{T} value may be obtained by using a first-order or second-order derivative method of determining the maximum of the first-order or second-order derivative curve of the sigmoid curve as the C_{T} value.

There remains a need for new methods and devices for performing PCR reactions.

### SUMMARY

Provided are methods and apparatuses for performing quantitative analysis of nucleic acid using real-time polymerase chain reaction (PCR).

Provided are computer readable recording media having recorded thereon computer programs for implementing the methods on computers.

Additional aspects will be set forth in part in the description which follows and, in part, will be apparent from the description, or may be learned by practice of the presented embodiments.

Provided is a method for performing quantitative analysis of a nucleic acid, the method including determining a curve-fitting area, including a cycle at which fluorescence intensity begins to increase exponentially, based on fluorescence intensity data obtained by performing PCR on a target nucleic acid; analyzing parameters for amplification efficiency and nucleic acid concentration by curve-fitting a result of performing PCR on a reference nucleic acid with a known initial nucleic acid concentration; and estimating the initial nucleic acid concentration of the target nucleic acid by performing curve-fitting on the determined curve-fitting area using the analyzed parameters.

According to another aspect of the present invention, provided is a computer-readable recording medium having recorded thereon a computer program for implementing the method for performing quantitative analysis of a nucleic acid on a computer.

According to another aspect of the present invention, provided is a nucleic acid quantitative analyzing apparatus including a curve-fitting area determining unit which determines a curve-fitting area including a cycle at which fluorescence intensity begins to increase exponentially, based on fluorescence intensity data obtained by performing PCR on a target nucleic acid; a parameter analyzing unit which analyzes parameters for amplification efficiency and nucleic acid concentration by curve-fitting a result of performing PCR on a reference nucleic acid with a known initial nucleic acid concentration; and a concentration estimating unit which estimates the initial nucleic acid concentration of the target nucleic acid by performing curve-fitting on the determined curve-fitting area using the analyzed parameters.

### BRIEF DESCRIPTION OF THE DRAWINGS

These and/or other aspects will become apparent and more readily appreciated from the following description of the embodiments, taken in conjunction with the accompanying drawings in which:

FIG. 1 is a block diagram of a nucleic acid quantitative analyzing apparatus according to an embodiment of the present invention;

FIG. 2 is a graph showing a PCR amplification curve for describing a curve-fitting area according to an embodiment of the present invention;

FIG. 3 is a diagram showing that a curve-fitting area determining unit determines a curve-fitting area according to an embodiment of the present invention;

FIG. 4 is a flowchart showing that a concentration estimating unit relatively estimates the initial nucleic acid concentration of a target nucleic acid, according to an embodiment of the present invention;

FIG. 5 is a flowchart showing a method that the concentration estimating unit absolutely estimates the initial nucleic acid concentration of a target nucleic acid, according to an embodiment of the present invention; and

FIG. 6 is a flowchart showing a method of performing quantitative analysis of a nucleic acid, according to an embodiment of the present invention.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description.

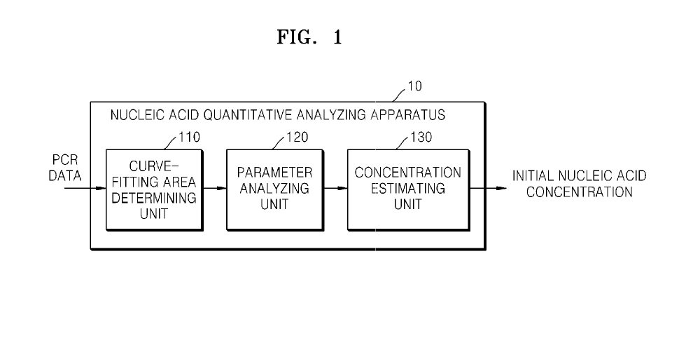

FIG. 1 is a block diagram of a nucleic acid quantitative analyzing apparatus 10 according to an embodiment of the present invention. Referring to FIG. 1, the nucleic acid quantitative analyzing apparatus 10 includes a curve-fitting area determining unit 100, a parameter analyzing unit 120, and a concentration estimating unit 130. To clearly describe the present embodiment, FIG. 1 only shows hardware components according to the present embodiment. However, one of ordinary skill in the art understands that the nucleic acid quantitative analyzing apparatus 10 according to the present embodiment may further include general-purpose hardware components other than the hardware components shown in FIG. 1.

Particularly, the nucleic acid quantitative analyzing apparatus 10 shown in FIG. 1 may be embodied as a processor. The processor may be formed of an array of a plurality of logic gates or a combination of a general-purpose microprocessor and a memory having stored therein programs to be executed on the microprocessor. Furthermore, one of ordinary skill in the art understands that the nucleic acid quantitative analyzing apparatus 10 according to the present embodiment may be embodied with other hardware than the one shown.

Real-time multiplex polymerase chain reaction (PCR) is one of the most frequently used methods from among various analyzing methods for detecting and quantifying nucleic acids from gene samples and is well-known in the art.

Briefly, a PCR apparatus (not shown) is an apparatus for amplifying nucleic acids by geometric progression via three stages including (1) a denaturation stage for separating nucleic acid strands from each other using heat, (2) an annealing stage in which the temperature is lowered and primers bind to an end of the nucleic acid sequence to be amplified, and (3) a polymerization (or extension) stage in which a polymerization reaction is induced to synthesize the nucleic acid. Particularly, PCR apparatuses widely use real-time PCR, which enables quantitative analysis of nucleic acids via real-time detection of intensities of fluorescent signals corresponding to the concentration of amplified nucleic acids.

The nucleic acid quantitative analyzing apparatus 10 according to the present embodiment may correspond to an apparatus for quantitative analysis of an initial concentration of nucleic acid based on PCR data, which is a result of performing such a real-time PCR. However, one of ordinary skill understands that any data regarding fluorescence intensity related to the amplification of nucleic acids other than real-time PCR data may be utilized.

Real-time PCR data are typically produced as a sigmoidal-shaped amplification plot in which fluorescence is plotted against the number of cycles. A cycle threshold (C_{T}) value serves as a tool for calculation of the initial concentration of the starting template in a sample (i.e., quantification of the initial concentration of nucleic acid in a sample). A C_{T} may be defined as a particular cycle number on the Sigmoid curve representing a PCR result. A C_{T} may be obtained by using a threshold value method of determining an x-axis value crossing the sigmoid curve representing fluorescence intensity by setting an arbitrary line parallel to the x-axis. Alternatively, a C_{T} value may be obtained by using a first-order or second-order derivative method of determining the maximum of the first-order or second-order derivative curve of the Sigmoid curve as the C_{T} value.

Existing methods assume that the efficiency of real-time PCR amplification efficiency is always constant. However, in reality, real-time PCR amplification efficiency changes over the course of real-time PCR according to the types of nucleic acids being amplified, the formation of inhibitors, the stabilities of reagents, etc. Furthermore, such a change in PCR amplification efficiency may cause the slope of a PCR amplification curve to change or shift. As a result, existing methods for quantitative analysis may involve a certain degree of quantitative error.

However, the nucleic acid quantitative analyzing apparatus 10 according to the present embodiment reflects the variability of PCR amplification efficiency due to outside environmental factors, such as the formation of an inhibitor, stabilities of reagents, etc. and, thus, quantitative analysis of initial concentrations of nucleic acids may be performed more accurately. Hereinafter, a configuration and operation of the nucleic acid quantitative analyzing apparatus 10 according to the present embodiment will be described in detail.

Referring back to FIG. 1, the curve-fitting area determining unit 110 determines a curve-fitting area including the cycle at which fluorescence intensity begins to increase exponentially, based on data obtained by performing real-time PCR on a target nucleic acid. The curve-fitting area corresponds to an area or region of the curve including fluorescence intensities at cycles nearby the cycle at which fluorescence intensity begins to increase exponentially.

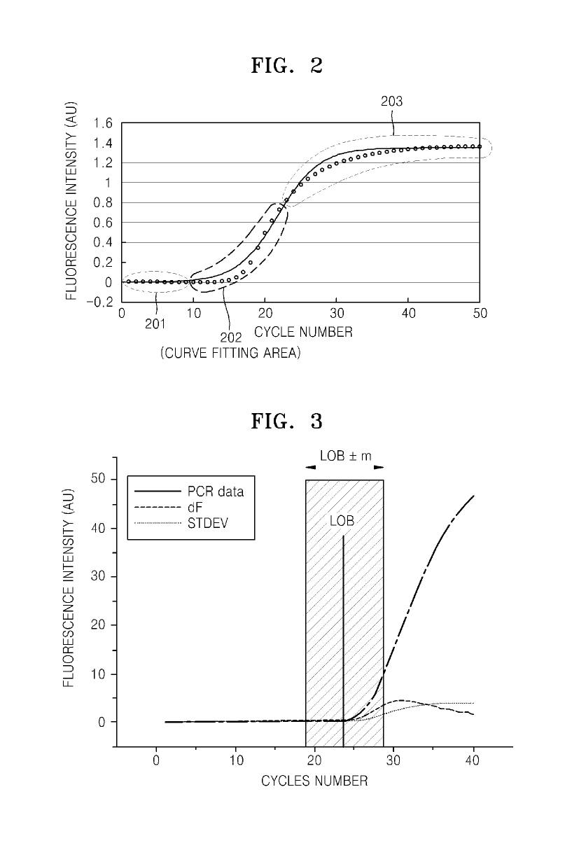

FIG. 2 is a graph showing a PCR amplification curve showing a curve-fitting area according to an embodiment of the present invention. Referring to FIG. 2, the PCR amplification curve is a graph of fluorescence intensity versus amplification cycle number.

As described above, a C_{T} value may be obtained from a PCR amplification curve. However, an area 201 corresponds to an area in which no fluorescence intensity or a little fluorescence intensity is detected due to insufficient amplification of a nucleic acid. Therefore, a fluorescence intensity in the area 201 may be data with insufficient precision for quantification of the initial concentration of the nucleic acid. Furthermore, an area 203 corresponds to an area in which the nucleic acid is significantly amplified. However, since the area 203 is affected by a number of variables, such as the formation of inhibitors due to the disintegration of reactants (dNTP, primers, probes, etc.), etc., a fluorescence intensity in the area 203 may also be data with insufficient precision for quantification of the initial concentration of the nucleic acid.

However, the curve-fitting area 202 according to the present embodiment is an area in which fluorescence intensity begins to be precisely detected and corresponds to an area not yet affected by the formation of inhibitors. Therefore, the curve-fitting area 202 corresponds to an area necessary for precise quantification of the initial concentration of the nucleic acid. The curve-fitting area determining unit 110 determines the curve-fitting area 202.

The curve-fitting area determining unit 110 compares the fluorescence intensities of adjacent cycles to a predetermined critical value and determines a cycle at which fluorescence intensity begins to increase exponentially. Here, the cycle at which fluorescence intensity begins to increase exponentially may correspond to a cycle obtained by using limit of blank (LOB). Since the LOB is obvious to one of ordinary skill in the art, a detailed description thereof is omitted.

First, the curve-fitting area determining unit 110 determines a cycle at which fluorescence intensity begins to increase exponentially, using Equation 1 below.

[Equation 1]

If dFₙ>Average(1:n−1)+STDEV(1:n−1)·Z,

dFₙ=fluorescence intensityₙ−fluorescence intensityₙ₋₁

n=LOB

In Equation 1, Average(1:n−1) denotes an average fluorescence intensity in the first through n−1^{th} cycles, STDEV(1:n−1) denotes an average deviation in fluorescence intensities in the first through n−1^{th} cycles, and Z denotes a variable according to a confidence interval of a blank distribution.

Here, in case of a 95% percentile of a blank distribution, the value of Z may be 1.645, for example. Furthermore, the value of Z may be between 1.645 and 3.291 according to percentiles of a set confidence interval. However, one of ordinary skill in the art understands that the value of Z is not limited thereto.

In Equation 1, if a dFₙ value for an n^{th} cycle exceeds a pre-set confidence interval (95%, 99%, etc.), it may be said that the dFₙ value has significantly deviated as compared to a previous dFₙ₋₁ value. Therefore, if a dFₙ value is found, the curve-fitting area determining unit 110 determines the n^{th} cycle as a cycle at which fluorescence intensity begins to increase exponentially.

Next, the curve-fitting area determining unit 110 determines m^{th} cycles (−7≦m≦7, m is an integer) around the cycle at which fluorescence intensity begins to increase exponentially (the n^{th} cycle). Here, m may be ±4 or ±5.

Next, the curve-fitting area determining unit 110 determines an area including fluorescence intensities corresponding to both the cycle at which fluorescence intensity begins to increase exponentially (the n^{th} cycle) and the determined nearby cycles (the m^{th} cycles on either side (±) of the n^{th} cycle) as a curve-fitting area.

As described above, the curve-fitting area, including the cycle at which fluorescence intensity begins to increase exponentially (the n^{th} cycle) and the m^{th} cycles around the n^{th} cycles, may be an area including a portion of a baseline area and the early phase of an exponential curve in the PCR amplification curve shown in FIG. 2.

FIG. 3 is a diagram showing that the curve-fitting area determining unit 110 determines a curve-fitting area according to an embodiment of the present invention. Referring to FIG. 3, as described above, the curve-fitting area determining unit 110 determines a curve-fitting area including LOB, that is, the cycle at which fluorescence intensity begins to increase exponentially (n^{th} cycle) and m^{th} cycles therearound.

Referring to FIG. 1, the parameter analyzing unit 120 analyzes parameters related to amplification efficiency and nucleic acid concentration by curve-fitting real-time PCR results to a reference with a known nucleic acid concentration.

The results of real-time PCR according to the present embodiment may be curve-fitted according to Equation 2 below.

[Equation 2]

F=δ·[DNA]₀·(1+E·V)ⁿ or

F=δ·[DNA]₀·(1+V·(1−V))ⁿ

In Equation 2, F denotes fluorescence intensity, δ is a constant indicating an efficiency of a PCR apparatus (not shown), [DNA]₀ denotes initial nucleic acid concentration, E denotes amplification efficiency, n denotes the number of PCR cycles, and V is a parameter according to an outside environmental factor affecting the amplification efficiency.

Here, δ·[DNA]₀ may be substituted with an arbitrary variable. For convenience of explanation, it will be assumed that δ·[DNA]₀ is 10̂a.

One of ordinary skill in the art understands that not only Equation 2, but also any equation derived therefrom or any other similar equation may be used in the present embodiment.

As described above, existing methods of quantitative analysis of a nucleic acid do not account for changes in amplification efficiency over the course of real-time PCR. However, in the present embodiment, a more precise quantitative analysis may be performed using parameters V and (a) related to amplification efficiency and nucleic acid concentration, respectively.

The amplification efficiency parameter (V) is a parameter for considering outside environmental factors affecting amplification efficiency, whereas the nucleic acid concentration parameter (a) is a parameter for considering outside environmental factors affecting the nucleic acid concentration.

The parameter analyzing unit 120 obtains a parameter V_{R} related to amplification efficiency and a parameter a_{R} related to nucleic acid concentration from performing real-time PCR on a reference nucleic acid with a known initial nucleic acid concentration [DNA]o according to Equation 2.

The concentration estimating unit 130 estimates the initial concentration of the nucleic acid by performing curve-fitting on the curve-fitting area, which is determined by the curve-fitting area determining unit 110, by using the parameters analyzed by the parameter analyzing unit 120.

In detail, the concentration estimating unit 130 corrects an amplification efficiency used for curve-fitting the determined curve-fitting area using the analyzed amplification efficiency parameter and corrects a nucleic acid concentration parameter a_{U} related to a target nucleic acid (or an unknown sample) obtained as a result of curve-fitting based on the corrected amplification efficiency using the nucleic acid concentration parameter a_{R} analyzed above. In other words, the initial nucleic acid concentration of the target nucleic acid estimated by the concentration estimating unit 130 is estimated based on such corrections.

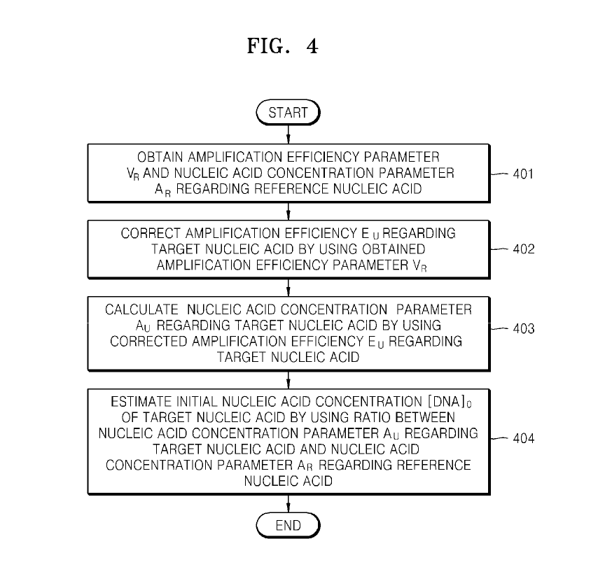

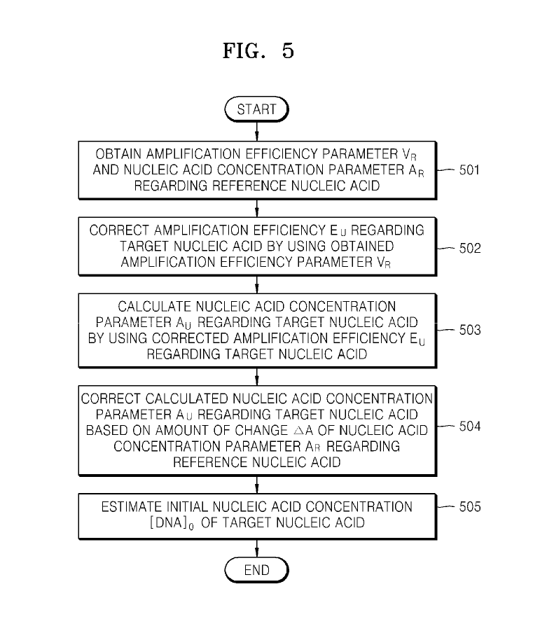

The concentration estimating unit 130 estimates the initial nucleic acid concentration of a target nucleic acid using a method of relative estimation as compared to a reference nucleic acid (FIG. 4) or a method of absolute estimation (FIG. 5).

FIG. 4 is a flowchart showing that the concentration estimating unit 130 estimates the initial nucleic acid concentration of a target nucleic acid, according to an embodiment of the present invention.

In operation 401, the concentration estimating unit 130 obtains the amplification efficiency parameter V_{R} and the nucleic acid concentration parameter a_{R} regarding the reference nucleic acid analyzed by the parameter analyzing unit 120.

In operation 402, the concentration estimating unit 130 corrects an amplification efficiency E_{U} regarding a target nucleic acid by using the obtained amplification efficiency parameter V_{R}. Here, the amplification efficiency E_{U} regarding the target nucleic acid may be corrected according to Equation 3 below.

[Equation 3]

E_{U}=E_{R}·V_{R}

In Equation 3, E_{U} denotes an amplification efficiency of a target nucleic acid, E_{R} denotes an amplification efficiency of a reference nucleic acid, and V_{R} denotes an amplification efficiency parameter related to the reference nucleic acid.

In operation 403, the concentration estimating unit 130 calculates the nucleic acid concentration parameter a_{U} of the target nucleic acid using the corrected amplification efficiency E_{U} of the target nucleic acid.

In operation 404, the concentration estimating unit 130 estimates the initial nucleic acid concentration [DNA]₀ of the target nucleic acid using a ratio between the nucleic acid concentration parameter a_{U} of the target nucleic acid and the nucleic acid concentration parameter a_{R} of the reference nucleic acid. Here, the initial nucleic acid concentration [DNA]₀ of the target nucleic acid may be calculated using the equation δ[DNA]₀=10̂a_{U} as described above.

FIG. 5 is a flowchart showing that the concentration estimating unit 130 the initial nucleic acid concentration of a target nucleic acid, according to an embodiment of the present invention.

In operation 501, the concentration estimating unit 130 obtains the amplification efficiency parameter V_{R} and the nucleic acid concentration parameter a_{R} of the reference nucleic acid analyzed by the parameter analyzing unit 120 as described above.

In operation 502, the concentration estimating unit 130 corrects the amplification efficiency E_{U} of a target nucleic acid using the obtained amplification efficiency parameter V_{R}. At this point, the amplification efficiency of the target nucleic acid may be corrected according to Equation 3 as described above.

In operation 503, the concentration estimating unit 130 calculates the nucleic acid concentration parameter a_{U} of the target nucleic acid using the corrected amplification efficiency E_{U} of the target nucleic acid.

In operation 504, the concentration estimating unit 130 corrects the calculated nucleic acid concentration parameter a_{U} of the target nucleic acid based on an amount of change Δa of the nucleic acid concentration parameter a_{R} of the reference nucleic acid. In other words, the concentration estimating unit 130 curve-shifts a result of performing curve-fitting on the target nucleic acid.

In operation 505, the concentration estimating unit 130 finally estimates the initial nucleic acid concentration [DNA]₀ of the target nucleic acid. At this point, an equation [DNA]₀=(10̂-a_{U})/δ that is obtained from the equation described above may be used for estimating the initial nucleic acid concentration [DNA]₀ of the target nucleic acid.

As described above, the nucleic acid quantitative analyzing apparatus 10 according to the present embodiment may analyze the initial concentration of a nucleic acid more accurately by reflecting the variability of the PCR amplification efficiency due to outside environmental factors in quantitative analysis of the nucleic acid.

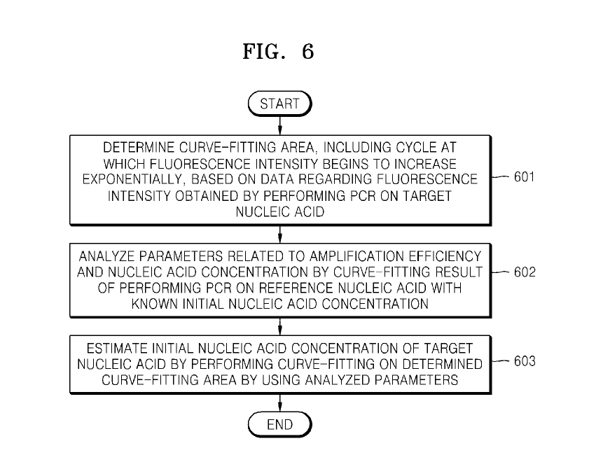

FIG. 6 is a flowchart showing a method of performing quantitative analysis of a nucleic acid, according to an embodiment of the present invention. Referring to FIG. 6, the method of performing quantitative analysis of a nucleic acid includes operations that are chronologically performed by the nucleic acid quantitative analyzing apparatus 10 shown in FIG. 1. Therefore, even if omitted below, any of the descriptions provided above with reference to FIG. 1 may also be applied to the method of method of performing quantitative analysis of a nucleic acid, according to the present embodiment.

In operation 601, the curve-fitting area determining unit 110 determines a curve-fitting area, including a cycle at which fluorescence intensity begins to increase exponentially, based on fluorescence intensity data obtained by performing real-time PCR on a target nucleic acid.

In operation 602, the parameter analyzing unit 120 analyzes parameters related to amplification efficiency and nucleic acid concentration by curve-fitting a result of performing PCR on a reference nucleic acid with a known initial nucleic acid concentration.

In operation 603, the concentration estimating unit 130 estimates the initial nucleic acid concentration of the target nucleic acid by performing curve-fitting on the determined curve-fitting area by using the analyzed parameters.

Processes, functions, methods, and/or software in apparatuses described herein may be recorded, stored, or fixed in one or more non-transitory computer-readable storage media (computer readable recording medium) that includes program instructions (computer readable instructions) to be implemented by a computer to cause one or more processors to execute or perform the program instructions. The media may also include, alone or in combination with the program instructions, data files, data structures, and the like. The media and program instructions may be those specially designed and constructed, or they may be of the kind well-known and available to those having skill in the computer software arts. Examples of non-transitory computer-readable storage media include magnetic media, such as hard disks, floppy disks, and magnetic tape; optical media such as CD ROM disks and DVDs; magneto-optical media, such as optical disks; and hardware devices that are specially configured to store and perform program instructions, such as read-only memory (ROM), random access memory (RAM), flash memory, and the like. Examples of program instructions include machine code, such as produced by a compiler, and files containing higher level code that may be executed by the computer using an interpreter. The described hardware devices may be configured to act as one or more software modules that are recorded, stored, or fixed in one or more computer-readable storage media, in order to perform the operations and methods described above, or vice versa. In addition, a non-transitory computer-readable storage medium may be distributed among computer systems connected through a network and computer-readable codes or program instructions may be stored and executed in a decentralized manner. In addition, the computer-readable storage media may also be embodied in at least one application specific integrated circuit (ASIC) or Field Programmable Gate Array (FPGA).

It should be understood that the exemplary embodiments described therein should be considered in a descriptive sense only and not for purposes of limitation. Descriptions of features or aspects within each embodiment should typically be considered as available for other similar features or aspects in other embodiments.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above- described elements in all possible variations thereof is encompassed by the invention unless otherwise indicated herein or otherwise clearly contradicted by context.

What is claimed is:

1. A method for performing quantitative analysis of a nucleic acid, the method comprising:
determining a curve-fitting area, including a cycle at which fluorescence intensity begins to increase exponentially, based on data regarding fluorescence intensity obtained by performing PCR on a target nucleic acid;
analyzing parameters related to amplification efficiency and nucleic acid concentration by curve-fitting a result of performing PCR on a reference nucleic acid with a known initial nucleic acid concentration; and
estimating the initial nucleic acid concentration of the target nucleic acid by performing curve-fitting on the determined curve-fitting area by using the analyzed parameters.

2. The method of claim 1, wherein the cycle at which fluorescence intensity begins to increase exponentially is determined by comparing a difference between fluorescence intensities of adjacent cycles to a predetermined critical value.

3. The method of claim 1, wherein the cycle at which fluorescence intensity begins to increase exponentially corresponds to a cycle obtained by using limit of blank (LOB).

4. The method of claim 1, wherein the determined curve-fitting area is an area including fluorescence intensities at cycles nearby the cycle at which fluorescence intensity begins to increase exponentially.

5. The method of claim 4, wherein the nearby cycles include m cycles around the cycle at which fluorescence intensity begins to increase exponentially (−7≦m≦7, m is an integer).

6. The method of claim 1, wherein the estimating of the initial nucleic acid concentration of the target nucleic acid comprises:
correcting an amplification efficiency used for performing curve-fitting on the determined curve-fitting area by using the analyzed parameter related to amplification efficiency; and
correcting a parameter related to nucleic acid concentration of the target nucleic acid, which is obtained as a result of performing curve-fitting based on the corrected parameter related to amplification efficiency, by using the analyzed parameter related to the nucleic acid concentration, and
the initial nucleic acid concentration of the target nucleic acid is estimated based on the results of the corrections.

7. The method of claim 1, wherein the parameter related to amplification efficiency is a parameter for considering outside environmental factors affecting a change of the amplification efficiency.

8. The method of claim 1, wherein the parameter related to nucleic acid concentration is a parameter for considering outside environmental factors affecting a change of quantity of nucleic acid concentration.

9. The method of claim 1, wherein the cycle at which fluorescence intensity begins to increase exponentially satisfies an equation, that is,
If dFₙ>Average(1:n−1)+STDEV(1:n−1)·Z,
dFₙ=fluorescence intensity_{n-fluorescence intensity}ₙ₋₁
n=LOB (Average(1:n−1)
denotes an average fluorescence intensity in first through n−1^{th} cycles, STDEV(1:n−1) denotes an average deviation in fluorescence intensities in first through n−1^{th} cycles, and Z denotes a variable according to a confidence interval of a blank distribution).

10. The method of claim 1, wherein the initial nucleic acid concentration of the target nucleic acid is estimated according to an equation, that is,
F=δ·[DNA]₀·(1+E·V)ⁿ or
F=δ·[DNA]₀·(1+E·(1−V))ⁿ
(F denotes fluorescence intensity, δ is a constant indicating an efficiency of a PCR device (, [DNA]₀ denotes an initial nucleic acid concentration, E denotes amplification efficiency, n denotes the number of PCR cycles, and V is a parameter according to an outside environmental factor affecting the amplification efficiency).

11. A non-transitory computer-readable recording medium having recorded thereon a computer program for implementing the method of claim 1 on a computer.

12. A nucleic acid quantitative analyzing apparatus comprising:
a curve-fitting area determining unit which determines a curve-fitting area including a cycle at which fluorescence intensity begins to increase exponentially, based on data regarding fluorescence intensity obtained by performing PCR on a target nucleic acid;
a parameter analyzing unit which analyzes parameters related to amplification efficiency and nucleic acid concentration by curve-fitting a result of performing PCR on a reference nucleic acid with a known initial nucleic acid concentration; and
a concentration estimating unit which estimates the initial nucleic acid concentration of the target nucleic acid by performing curve-fitting on the determined curve-fitting area by using the analyzed parameters.

13. The acid quantitative analyzing apparatus of claim 12, wherein the cycle at which fluorescence intensity begins to increase exponentially is determined by comparing a difference between fluorescence intensities of adjacent cycles to a predetermined critical value.

14. The acid quantitative analyzing apparatus of claim 12, wherein the cycle at which fluorescence intensity begins to increase exponentially corresponds to a cycle obtained by using limit of blank (LOB).

15. The acid quantitative analyzing apparatus of claim 12, wherein the determined curve-fitting area is an area including fluorescence intensities at cycles nearby the cycle at which fluorescence intensity begins to increase exponentially.

16. The acid quantitative analyzing apparatus of claim 15, wherein the nearby cycles include m cycles around the cycle at which fluorescence intensity begins to increase exponentially (−7≦m≦7, m is an integer).

17. The acid quantitative analyzing apparatus of claim 12, wherein the concentration estimating unit corrects an amplification efficiency used for performing curve-fitting on the determined curve-fitting area by using the analyzed parameter related to amplification efficiency and corrects a parameter related to nucleic acid concentration of the target nucleic acid, which is obtained as a result of performing curve-fitting based on the corrected parameter related to amplification efficiency, by using the analyzed parameter related to the nucleic acid concentration, and
the initial nucleic acid concentration of the target nucleic acid is estimated based on the results of the corrections.

18. The acid quantitative analyzing apparatus of claim 12, wherein the parameter related to amplification efficiency is a parameter for considering outside environmental factors affecting a change of the amplification efficiency.

19. The acid quantitative analyzing apparatus of claim 12, wherein the parameter related to nucleic acid concentration is a parameter for considering outside environmental factors affecting a change of quantity of nucleic acid concentration.
